(19) United States
(12) Patent Application Publication
(10) Pub. No.: US 2025/0032794 A1
(43) Pub. Date: Jan. 30, 2025

(54) STIMULATION DEVICE AND METHODS OF STIMULATION

(71) Applicant: THE ALFRED E. MANN FOUNDATION FOR SCIENTIFIC RESEARCH, Valencia, CA (US)

(72) Inventor: Christopher Reed Jenney, Santa Clarita, CA (US)

(21) Appl. No.: 18/782,017

(22) Filed: Jul. 23, 2024

Related U.S. Application Data
(60) Provisional application No. 63/515,306, filed on Jul. 24, 2023.

(51) Int. Cl. A61N 1/36; A61N 1/05; A61N 1/372; A61N 1/378

(52) U.S. Cl. CPC A61N 1/3611; A61N 1/0504; A61N 1/36139; A61N 1/37217; A61N 1/3787

(57) ABSTRACT

A stimulation system includes: a mouthpiece including one or more sensors to intraorally sense physiological data of a subject; and a subcutaneously implantable device to be in communication with the mouthpiece and including one or more stimulators to stimulate at least one of a nerve or a muscle of the subject to treat apnea.

### CROSS-REFERENCE TO RELATED APPLICATION

This application claims priority to, and the benefit of, U.S. Provisional Patent Application No. 63/515,306, filed on Jul. 24, 2023, the entire content of which is hereby incorporated by reference.

### BACKGROUND

### 1. Field

The present disclosure relates to systems and methods for providing stimulation to treat sleep apnea and/or one or more other medical conditions.

### 2. Description of the Related Art

Stimulation devices are utilized to stimulate various suitable muscles and/or nerves (e.g., hypoglossal nerve) to treat sleep apnea and other medical conditions. Stimulation devices that are implanted subcutaneously can provide more precise, consistent, and efficient control of muscles that control a subject's tongue and/or airway as compared to intraoral stimulation devices. In some instances, it may be desirable to provide stimulation in response to an apneic event, and physiological data indicative of an apneic event. Subcutaneously implanted devices, however, fail to accurately detect the occurrence of apneic events. Therefore, there is a desire to improve stimulation devices for treating medical conditions such as sleep apnea so that the stimulation device may both precisely, consistently, and efficiently control subject's tongue and/or airway, and reliably provide the stimulation in response to precisely detecting occurrence of the apneic events.

Statements provided in this Background section are provided to explain background information relating to, and useful for understanding, the present disclosure, and are not admissions of prior art.

### SUMMARY

Aspects of embodiments of the present disclosure are directed toward systems and methods for providing stimulation to treat apnea and/or one or more other medical conditions.

Additional aspects will be set forth in part in the description which follows and, in part, will be apparent from the description, or may be learned by practice of the presented embodiments of the disclosure.

According to an embodiment, a stimulation system includes: a mouthpiece including one or more sensors to intraorally sense physiological data of a subject (e.g., patient); and a subcutaneously implantable device to be in communication with the mouthpiece and including one or more stimulators to stimulate a hypoglossal nerve of the subject to treat sleep apnea and/or one or more other medical conditions.

In an embodiment, the one or more sensors include at least one of an ultrasound sensor, a moisture sensor, a temperature sensor, an inertial sensor, an electrocardiogram sensor, a pulse oximeter, an airflow sensor, a microphone, a pressure sensor, an actigraphy sensor, or an electroencephalogram sensor.

In another embodiment, the one or more sensors include at least one of an ultrasound sensor, a moisture sensor, a temperature sensor, an inertial sensor, or an electrocardiogram sensor.

In another embodiment, the one or more sensors include at least two of an ultrasound sensor, a moisture sensor, a temperature sensor, an inertial sensor, an electrocardiogram sensor, a pulse oximeter, an airflow sensor, a microphone, a pressure sensor, an actigraphy sensor, or an electroencephalogram sensor.

In another embodiment, the mouthpiece includes a transmitter to wirelessly transmit a communication signal based on the physiological data sensed by the one or more sensors, and the subcutaneously implantable device includes a receiver to wirelessly receive the communication signal.

In another embodiment, the mouthpiece includes: a battery; and a controller to determine, based on the physiological data, that an apneic event of the subject has occurred, and to cause the receiver to transmit the communication signal as a stimulation-start signal in response to determining that the apneic event has occurred.

In another embodiment, the mouthpiece is to transmit the communication signal as a data signal including at least some of the physiological data, and the subcutaneously implantable device includes a controller to determine, based on the physiological data, that an apneic event of the subject has occurred and to stimulate the nerve or the muscle in response to determining that the apneic event has occurred.

In another embodiment, the one or more stimulators are to stimulate at least a hypoglossal nerve.

In another embodiment, the subcutaneously implantable device includes a housing including one or more electronic components, and the one or more stimulators includes a stimulator lead extending, at a proximal end of the stimulator lead, from the housing and a cuff electrode positioned along the stimulator lead and to stimulate the hypoglossal nerve.

In another embodiment, the subcutaneously implantable device includes a battery to provide power to the one or more stimulators.

In another embodiment, the subcutaneously implantable device includes: a receiver to wirelessly receive a communication signal from the mouthpiece; and a controller communicatively coupled to the receiver and to cause power to be provided from the battery to the one or more stimulators in response to the receiver receiving the communication signal.

According to an embodiment, a stimulation system includes: a subcutaneously implantable device including: one or more stimulators to provide nerve stimulation to a hypoglossal nerve of a subject; a battery to provide power to the one or more stimulators; and a receiver device to wirelessly receive a communication signal, wherein the subcutaneously implantable device is to provide the nerve stimulation, via the one or more stimulators, based on the communication signal.

In an embodiment, the subcutaneously implantable device includes a controller communicatively coupled to the receiver device and to controllably cause the power to be provided from the battery to the one or more stimulators.

In another embodiment, the communication signal is a data signal including physiological data, and the controller is to cause the power to be provided from the battery to the one or more stimulators in response to determining, by the controller and based on the physiological data, that an apneic event of the subject has occurred.

In another embodiment, the communication signal is a stimulation-start signal, and the controller is to cause the power to be provided from the battery to the one or more stimulators in response to the receiver device receiving the stimulation-start signal.

In another embodiment, the stimulation-start signal is a qualified stimulation-start signal that the controller is to recognize as being generated by a qualified mouthpiece device.

In another embodiment, the subcutaneously implantable device includes a housing including the battery and the receiver device, and the one or more stimulators includes a stimulator lead extending, at a proximal end of the stimulator lead, from the housing and one or more electrodes positioned along the stimulator lead and to stimulate the hypoglossal nerve.

In another embodiment, the medical stimulation system includes a mouthpiece including one or more sensors to intraorally sense physiological data of the subject.

In another embodiment, the mouthpiece includes a transmitter to wirelessly transmit the communication signal to the receiver device of the subcutaneously implantable device.

In another embodiment, the mouthpiece includes a controller to determine, based on the physiological data, whether an apneic event of the subject has occurred, and to transmit, by the transmitter, a stimulation-start signal to the receiver device in response to determining that the apneic event has occurred.

In another embodiment, the one or more sensors include at least one of an ultrasound sensor, a moisture sensor, a temperature sensor, an inertial sensor, or an electrocardiogram sensor.

According to an embodiment, a method of providing stimulation for treating apnea includes: wirelessly receiving, by a receiver of a subcutaneously implanted device, a communication signal; and providing stimulation, by one or more stimulators of the subcutaneously implanted device and based on the communication signal, to a hypoglossal nerve of a subject.

In an embodiment, the method includes causing, by a controller of the subcutaneously implanted device and based on the communication signal, power to be provided by a battery of the subcutaneously implanted device to the one or more stimulators to provide the stimulation.

In another embodiment, the communication signal is a data signal including physiological data, and the method includes determining, by the controller and based on the physiological data, that an apneic event of the subject has occurred, and providing the stimulation in response to the determining that the apneic event has occurred.

In another embodiment, the communication signal is a stimulation-start signal, and the method includes providing the stimulation in response to the subcutaneously implanted device receiving the stimulation-start signal.

In another embodiment, the method includes intraorally sensing, by one or more sensors of a mouthpiece, physiological data of the subject.

In another embodiment, the method includes wirelessly transmitting, by a transmitter of the mouthpiece, the communication signal.

In another embodiment, the method includes determining, by a controller of the mouthpiece and based on the physiological data, that an apneic event of the subject has occurred, and transmitting the communication signal as a stimulation-start signal in response to the controller determining that the apneic event has occurred.

In another embodiment, the physiological data includes at least one data of an ultrasound measurement, a moisture measurement, a temperature measurement, an inertia measurement, an electrocardiogram, an oxygen-blood level, an airflow measurement, a sound measurement, a pressure measurement, an actigraphy-based measurement, or a brain activity measurement.

In another embodiment, the physiological data includes at least one data of an ultrasound measurement, a moisture measurement, a temperature measurement, an inertia measurement, or an electrocardiogram.

In another embodiment, the physiological data includes at least two data of an ultrasound measurement, a moisture measurement, a temperature measurement, an inertia measurement, an electrocardiogram, an oxygen-blood level, an airflow measurement, a sound measurement, a pressure measurement, an actigraphy-based measurement, or a brain activity measurement.

The example embodiments described in this summary section are nonlimiting and non-exhaustive.

### BRIEF DESCRIPTION OF THE DRAWINGS

The drawings, which form a part of this specification, illustrate nonlimiting and non-exhaustive example embodiments of the present disclosure.

FIG. 1 depicts a stimulation system according to some embodiments.

FIG. 2 depicts the stimulation system of FIG. 1 in use by a subject.

FIGS. 3, 4, and 5 are perspective views, each depicting a mouthpiece of a stimulation system according to some embodiments.

FIG. 6 depicts a method for providing stimulation to a hypoglossal nerve according to some embodiments.

FIG. 7 depicts a method for providing stimulation to a hypoglossal nerve according to some embodiments.

### DETAILED DESCRIPTION

Example embodiments of the present disclosure will now be described with reference to the accompanying drawings. In the drawings, the same or similar reference numerals refer to the same or similar elements throughout. As utilized herein, the use of the term “may,” when describing embodiments of the present disclosure, refers to “one or more embodiments of the present disclosure.” In addition, the use of alternative language, such as “or,” when describing embodiments of the present disclosure, refers to “one or more embodiments of the present disclosure” for each corresponding item listed.

The terminology utilized herein is for the purpose of describing particular embodiments only and is not intended to be limiting of the present disclosure. As utilized herein, the singular forms “a”, “an” and “the” are intended to include the plural forms as well, unless the context clearly indicates otherwise. It will be further understood that the terms “comprises,” “comprising,” “includes,” and “including,” when utilized in this specification, specify the presence of stated features, integers, steps, operations, elements, and/or components, but do not preclude the presence or addition of one or more other features, integers, steps, operations, elements, components, and/or groups thereof. As utilized herein, the term “and/or” includes any and all combinations of one or more of the associated listed items. Expressions such as “at least one of,” when preceding a list of elements, modify the entire list of elements and do not modify the individual elements of the list.

It will be understood that when an element is referred to as being “on”, “connected to”, “coupled to”, or “adjacent to” another element or layer, it can be directly on, connected to, coupled to, or adjacent to the other element, or one or more intervening element(s) may be present. In contrast, when an element is referred to as being “directly on,” “directly connected to”, “directly coupled to”, or “immediately adjacent to” another element, there are no intervening elements or layers present.

Nonlimiting and non-exhaustive example embodiments of systems and methods for providing stimulation will now be described with reference to the drawings.

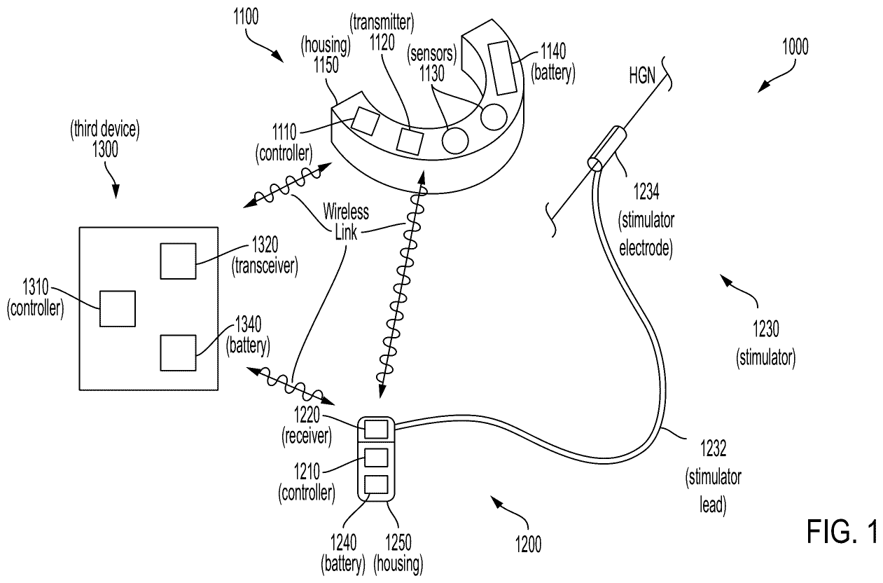

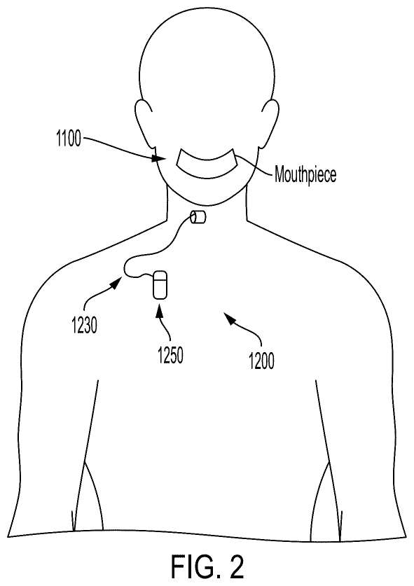

FIG. 1 depicts a stimulation system 1000 (e.g., an electrical stimulation system) according to some embodiments. FIG. 2 depicts the stimulation system 1000 of FIG. 1 in use by a subject.

Referring concurrently to FIGS. 1 and 2, the stimulation system 1000 may include a mouthpiece 1100 and an implantable device 1200 (e.g., subcutaneously implantable device). In some embodiments, the mouthpiece 1100 is configured to sense (e.g., measure) one or more kinds (or types) of physiological data and is to be in communication with the implantable device 1200, and the implantable device 1200 may be configured to provide stimulation to a subject based on the physiological data sensed by the mouthpiece 1100 in order to treat apnea and/or one or more other medical conditions (e.g., bruxism or one or more medical conditions related to apnea).

The subject may be any suitable subject, including a human or a non-human animal (e.g., a dog, a horse, etc.). Although the stimulation system 1000 will be described hereinafter with reference to use in a human, the present disclosure is not limited thereto.

In some embodiments, the stimulation system 1000 is to (e.g., configured to) provide stimulation to at least the hypoglossal nerve (HGN), for example, to only the HGN or to the HGN and/or to one or more other nerves and/or one or more muscles. The one or more other nerves and/or one or more muscles may control the tongue and/or airway of the subject. Stimulating the HGN may move the subject's tongue and/or cause the subject's airway to open so that the subject can breathe. This can be desirable when the subject is experiencing an apneic event or other medical condition where the subject's airway involuntarily (or unintentionally) closes or involuntarily (or unintentionally) remains closed. Although the stimulation system 1000 may be described hereinafter as being to stimulate the HGN, the present disclosure is not limited thereto.

The mouthpiece 1100 may be configured to be positioned at least partly (e.g., partly or entirely) intraorally in the subject's mouth. For example, the mouthpiece 1100 may be configured to be securely positioned on the roof and/or on the bottom of the subject's mouth. The mouthpiece 1100 may be configured (e.g., shaped and sized) to fit around the outside and/or around the inside of the bottom teeth of the subject and/or around the outside and/or around the inside of the top teeth of the subject. For example, the mouthpiece 1100 may be configured to fit at least partially over the bottom teeth and/or at least partially over the top teeth of the subject.

In some embodiments, the mouthpiece 1100 may be a unitary or single piece. In some other examples, the mouthpiece 1100 may include two or more parts (e.g., separate parts), for example, a first part configured to be securely positioned on the bottom of the subject's mouth and a second part (e.g., a separate second part) configured to be securely positioned on the roof of the subject's mouth. In some embodiments where the mouthpiece 1100 includes two or more separate parts, at least some of the two or more separate parts may be to be (e.g., configured to be) in communication with each other, for example, via a transmitter (e.g., wireless communication transmitter) and a receiver (e.g., wireless communication receiver). The mouthpiece 1100 may be configured to be intraorally installable and removable, for example, by a friction fit and/or without utilizing installation tools. For example, in some embodiments, the mouthpiece 1100 is not configured to be permanently installed in the subject's mouth.

The mouthpiece 1100 may be configured to sense (e.g., measure) one or more kinds of physiological data. For example, the mouthpiece 1100 may be configured to intraorally sense the physiological data. The mouthpiece 1100 may include a housing 1150 and one or more sensors 1130 on and/or at least partially within the housing 1150 (e.g., partially embedded within the housing 1150 and partially exposed, or entirely embedded within the housing 1150) and configured to sense one or more kinds of physiological data. The one or more sensors 1130 may, for example, be at least partially embedded within the housing 1150 and, in some examples, be exposed to the outside of the housing 1150 and/or protrude outwardly from the housing 1150.

The one or more sensors 1130 may be configured to sense (e.g., measure) at least one data (e.g., one data, two data, three data, four data, etc.) of an ultrasound measurement, a moisture measurement, a temperature measurement, an inertia measurement, an electrocardiogram, an oxygen-blood level, an airflow measurement, a sound measurement, a pressure measurement, an actigraphy-based measurement, a brain activity measurement, or a compression measurement. For example, the one or more sensors 1130 may be configured to sense at least one data of an ultrasound measurement, a moisture measurement, a temperature measurement, an inertia measurement, or an electrocardiogram. In some embodiments, the one or more sensors 1130 are configured to sense at least two data (e.g., two data, three data, four data, etc.) of an ultrasound measurement, a moisture measurement, a temperature measurement, an inertia measurement, an electrocardiogram, an oxygen-blood level, an airflow measurement, a sound measurement, a pressure measurement, an actigraphy-based measurement, or a brain activity measurement.

The one or more sensors 1130 may include at least one (e.g., one, two, three, four, etc.) of an ultrasound sensor, a moisture sensor, a temperature sensor, an inertial sensor (e.g., an inertial measurement unit, a gyroscope or micro-gyroscope, etc.), an electrocardiogram sensor, a pulse oximeter, an airflow sensor, a microphone, a pressure sensor, an actigraphy sensor, an electroencephalogram sensor, or a compression sensor. For example, the one or more sensors 1130 may include at least one of an ultrasound sensor (e.g., a miniature ultrasound transducer), a moisture sensor, a temperature sensor, an inertial sensor, or an electrocardiogram sensor. In some examples, the one or more sensors include at least two (e.g., two, three, four, etc.) of an ultrasound sensor, a moisture sensor, a temperature sensor, an inertial sensor, an electrocardiogram sensor, a pulse oximeter, an airflow sensor, a microphone, a pressure sensor, an actigraphy sensor, or an electroencephalogram sensor.

The physiological data that the one or more sensors 1130 may be configured to sense may indicate or be utilized (e.g., analyzed) to determine that an apneic event or other medical event (e.g., a medical event related to apnea, for example, a medical event often accompanying an apneic event) has occurred in any suitable manner as would be recognized by those skilled in the art. As utilized herein, an apneic event may refer to an occurrence where the subject's airway involuntarily (or unintentionally) closes or involuntarily (or unintentionally) remains closed for longer than a set period of time.

For example, a sound measurement by a microphone that is below a set value or below the set value for longer than a set time period may indicate below normal airflow, and thus, may indicate an apneic event. The sound measurement may detect sounds from the heart (e.g., slow heartbeat, no heartbeat, or irregular heartbeat) that indicate that an apneic event has occurred. The sound measurement may detect snoring, which can indicate continued airflow of the subject. An ultrasound measurement by an ultrasound transducer may be utilized to evaluate the position of the subject's tongue and/or collapse of the subject's airway, which can be utilized to determine that an apneic event has occurred. A moisture measurement by a moisture sensor that indicates an amount of saliva above a set amount can indicate that the subject's airway has closed, leading to a buildup of saliva. A temperature measurement by a temperature sensor that is above or below a threshold value may indicate low airflow, and thus, may indicate an apneic event.

An inertia measurement by an inertia sensor may determine the sleeping position (e.g., on back, on side, or on stomach) of the subject, which may be utilized to determine whether an apneic event is more or less likely. The inertia sensor may be configured to detect (e.g., measure) vibration (e.g., vibration associated with snoring), which can indicate snoring as a precursor to an apneic event. An electrocardiogram by an electrocardiogram sensor may be utilized to determine that a cardiac event (e.g., cardiac arrest) has occurred, which may be associated with (e.g., caused by) an apneic event. An oxygen-blood level measured by a pulse oximeter may detect low oxygen levels and/or low pulse rate, which may indicate a decrease in airflow and an apneic event. An airflow measurement by an airflow sensor below a set value or below a set value for longer than a set time period may indicate that the subject has stopped breathing, and thus, that an apneic event has occurred.

A pressure measurement by a pressure sensor may indicate that the subject is clenching and/or grinding his or her teeth, which can commonly occur together with an apneic event. In some examples, the pressure sensor is configured to be sensitive enough to detect (e.g., identify) breathing, lack of breathing, and/or movement of the tongue. An actigraphy-based measurement by an actigraphy sensor may indicate movement by the subject, which may indicate that the subject is breathing normally. The actigraphy-based measurement may also indicate the sleeping position of the subject, which may be utilized to determine whether an apneic event is more or less likely. A brain activity measurement by an electroencephalogram sensor may indicate one or more suitable sleep modes of the subject, which may be utilized to determine whether the subject is experiencing an apneic event. A compression measurement by a compression sensor may indicate whether the subject is grinding his or her teeth (e.g., while sleeping), which occurs in individuals who have bruxism. These example measurements and how they may be utilized are provided as examples, and the present disclosure is not limited thereto.

The mouthpiece 1100 may be configured to sense a plurality of different kinds of physiological data, and the determination, based on the physiological data, of whether an apneic event or other medical event has occurred may be based on only one or on multiple kinds of physiological sensed by the mouthpiece 1100. For example, each kind or kind of physiological data that is sensed may be analyzed to determine a respective likelihood of an apneic event based on the corresponding kind or kind of physiological data, and the plurality of respective likelihoods may be weighed together in a multi-factor analysis. However, this is just an example, and the present disclosure is not limited thereto.

The mouthpiece 1100 may include a mouthpiece controller 1110, a transmitter 1120 (e.g., a wireless communication transmitter), and a battery 1140 (e.g., a permanent or rechargeable battery), each of which may be at least partially within the housing 1150. The battery 1140 may be coupled (e.g., electrically coupled), and configured to provide power, to one or more components of the mouthpiece 1100, for example, to the one or more sensors 1130, the mouthpiece controller 1110, and/or the transmitter 1120. The transmitter 1120 may be configured to transmit (e.g., wirelessly transmit) a communication signal, for example, to the implantable device 1200 (e.g., to a receiver of the implantable device 1200) and/or to a third device (e.g., to a receiver of a third party device). The controller 1110 may be coupled (e.g., operatively coupled) to at least some of the components of the mouthpiece 1100 (e.g., to the one or more sensors 1130, the transmitter 1120, and/or the battery 1140) and configured, in response to executing computer-readable instructions stored in a memory of the mouthpiece 1100, to control at least some operations of the at least some of the components of the mouthpiece 1100. For example, the controller 1110 may be configured to controllably cause the sensors 1130 to start and stop sensing data, and to controllably cause the transmitter 1120 to transmit the communication signal.

In some embodiments, the controller 1110 is configured, in response to executing computer-readable instructions stored in the memory of the mouthpiece 1100, to cause the transmitter 1120 to transmit the communication signal as a data signal that includes at least some of the sensed (e.g., measured) physiological data. For example, the mouthpiece controller 1110 may cause the transmitter 1120 to continuously, or periodically, transmit the data signal as the physiological data is sensed by the one or more sensors 1130. The transmitted data signal may be received by the implantable device 1200, and the implantable device 1200 may be configured to determine, based on the physiological data of the received data signal and in response to a stimulator controller 1212 of the implantable device 1200 executing computer-readable instructions stored in a memory of the implantable device 1200, that an apneic event or other medical event has occurred. As described in more detail below, the implantable device 1200 may provide stimulation to the one or more muscles and/or the one or more nerves in response to determining that the apneic event or other medical event has occurred.

In some other examples, the mouthpiece controller 1110 is configured, in response to executing computer-readable instructions stored in the memory, to determine, based on the sensed physiological data (e.g., by analyzing the physiological data), that an apneic event or other medical event has occurred. In response to determining that the apneic event or other medical event have occurred, the mouthpiece controller 1110 may be configured, in response to executing computer-readable instructions stored in the memory, to cause the transmitter 1120 to transmit the communication signal as a stimulation start signal. As described in more detail below, the implantable device 1200 may provide stimulation to the one or more muscles and/or the one or more nerves in response to receiving the stimulation start signal. For example, in some embodiments the stimulation start signal does not include the physiological data sensed by the one or more sensors 1130, and the stimulator controller 1212 does not (e.g., is not configured to) analyze data of the stimulation start signal. Rather, the stimulator controller 1212 may be configured to determine whether the stimulation start signal has been received (e.g., by a stimulator receiver 1214 of the implantable device 1200) and to cause one or more stimulators 1230 of the implantable device 1200 to provide stimulation in response to determining that the stimulation start signal has been received.

Analyzing the physiological data to determine whether an apneic or another medical event has occurred can be relatively complex and require a relatively large amount of power. Accordingly, the required complexity and cost of the stimulator controller 1212 can be reduced, and the size and capacity of a stimulator battery 1216 (and thus the size of the implantable device 1200) can be reduced, by utilizing the mouthpiece 1100 to analyze the physiological data compared to utilizing the implantable device 1200 to analyze the physiological data. By reducing the size of the implantable device 1200, the invasiveness of the implantable device 1200 in the subject can be reduced, and the risk of complications during and after implanting the implantable device 1200 into the subject can be reduced.

In some other embodiments, the transmitter 1120 of the mouthpiece 1100 may be to transmit the data signal to a third device 1300, which may be a part of the stimulation system 1000, and may be configured to be in wireless communication with (e.g., to be wirelessly communicatively coupled or couplable to) the mouthpiece 1100 and the implantable device 1200. In some embodiments, the third device 1300 is a patient controller that is configured to control at least some operations of the mouthpiece 1100 and/or at least some operations of the implantable device 1200.

The third device 1300 may include a transceiver 1320 configured to receive communication signals (e.g., from the mouthpiece 1100) and to transmit communication signals (e.g., to the implantable device 1200), a controller 1310 configured to control at least some of the operations of the third device 1300, and a battery 1340 (e.g., a permanent or rechargeable battery) configured to provide power to components of the third device 1300. The third device 1300 may also have a user interface configured to provide information (e.g., via a display and/or a speaker) and to receive input from a subject. The controller 1310 of the third device 1300 may be configured, in response to executing computer-readable instructions stored in a memory of the third device 1300, to determine, based on the physiological data included in the received data signal, that an apneic event or another medical event have occurred. The controller 1310 may be configured, in response to executing computer-readable instructions stored in the memory of the third device 1300, to cause the transceiver 1320 to transmit a stimulation start signal to the implantable device 1200.

The implantable device 1200 may be configured to be at least partly (e.g., partly or entirely) subcutaneously implantable within the subject. For example, the implantable device 1200 may be implantable within the tissue of the subject (e.g., under the subject's skin), and may be implanted via cutting into the tissue of the subject and inserting the implantable device 1200 into the tissue of the subject.

The implantable device 1200 may be configured to provide stimulation (e.g., electrical stimulation) to one or more muscles and/or to one or more nerves to treat apnea and/or one or more other medical conditions (e.g., bruxism). In some embodiments, the implantable device 1200 is configured to provide the stimulation based on the physiological data sensed by the one or more sensors 1130 of the mouthpiece 1100.

The implantable device 1200 may include one or more stimulators 1230 (e.g., electrical stimulators), a receiver 1220 (e.g., a wireless communication receiver), a stimulator controller 1210, and a stimulator battery 1240 (e.g., a permanent or rechargeable battery). In some embodiments where the stimulator battery 1240 is a rechargeable battery, the implantable device 1200 may include a power receiver coil electrically coupled to the rechargeable stimulator battery 1240 and configured to wirelessly (e.g., inductively) receive power and to charge the rechargeable stimulator battery 1240 with the received power. In some embodiments, the implantable device 1200 includes a housing 1250, and the stimulator controller 1210, the receiver 1220, and the stimulator battery 1240 may be within (e.g., encased within) the housing 1250.

The housing 1250 may include (e.g., be) a material that is an electrical insulator or conductor (e.g., titanium). In some examples, the housing 1250 may be configured for in-vivo implantation. For example, the housing 1250 may be a hermetically sealed housing 1250, which can protect electronic components within the housing 1250 from being damaged by biological fluids around (e.g., surrounding) the housing 1250, and can protect the tissue around (e.g., surrounding) the housing 1250 from being damaged by electric currents of the electronic components. By configuring the housing 1250 to be hermetically sealed and/or by including a material that is configured for in-vivo implantation (e.g., a material that is biologically inert or biologically non-reactive), unintended and/or undesirable interactions between the housing 1250 and the tissue around (e.g., surrounding) the housing 1250 can be prevented or reduced. For example, the housing 1250 may include (e.g., as an outer surface), a bioinert metal, ceramic, glass, and/or polymer.

In some embodiments, the one or more stimulators 1230 are configured to provide stimulation (e.g., electrical stimulation) to tissue (e.g., biological tissue) in proximity to the one or more stimulators 1230. The one or more stimulators 1230 may be electrically coupled or couplable to the stimulator battery 1240, and the stimulator battery 1240 may be configured to provide power to the one or more stimulators 1230 to provide the stimulation. In some embodiments, the controller 1210 is configured, in response to executing computer readable instructions stored in the memory of the implantable device 1200, to control the power provided from the stimulator battery 1240 to the one or more stimulators 1230 to control the stimulation provided by the one or more stimulators 1230. For example, the stimulator controller 1210 may be configured to controllably (e.g., selectively) allow and block power to be provided from the stimulator battery 1240 to the one or more stimulators 1230 to respectively controllably start and stop the stimulation.

In some embodiments, the stimulator controller 1210 includes, and is configured to control, one or more switches configured to direct a voltage or electric current from a voltage/current source (e.g., a voltage/current source electrically coupled to the stimulator battery 1240) to the one or more stimulators 1230 (e.g., to an electrode of the stimulator 1230). The implantable device 1200 may include electronics (e.g., a current source and corresponding power modulation electronics), and the stimulator controller 1210 may be configured to control such electronics to generate a stimulation signal, having a set (e.g., predetermined) waveform and frequency, and to transmit the stimulation signal to a stimulator (e.g., to the stimulator electrode 1234).

In some other examples, the implantable device 1200 does not include a battery configured to provide power to the one or more stimulators 1230, and the one or more stimulators 1230 are powered by a wireless power transfer device that can be positioned outside of the subject and configured to inductively provide power to the one or more stimulators 1230 to provide the stimulation. In some such embodiments, although the size of the implantable device 1200 may be reduced by omitting the battery of the implantable device 1200, the complexity and cost of the stimulation system 1000 is increased by requiring an additional component (the wireless power transfer device), and the reliability of the power supply to the one or more stimulators 1230 may be reduced by relying on a wireless transfer of power instead of an electrical connection between the stimulator battery 1240 and the one or more stimulators 1230.

The one or more stimulators 1230 may be coupled to (e.g., electrically coupled to) the housing 1250 and may extend or protrude from the housing 1250, or may be at least partially encased within the housing 1250 and exposed to the outside of the housing 1250. The one or more stimulators 1230 may include any suitable number, and suitable kind, of stimulators for providing stimulation to the one or more muscles and/or the one or more nerves to treat apnea or the one or more other medical conditions. In some embodiments, the one or more stimulators 1230 includes a stimulator lead 1232 and one or more electrodes positioned along the stimulator lead 1232, and the one or more electrodes are configured (e.g., shaped, sized, and/or of a number) to stimulate the one or more muscles and/or the one or more nerves, for example, to stimulate the HGN. Because the shape, size, and position of muscles and nerves varies, the shape, size, and/or number of the one or more electrodes along the stimulator lead 1232 may vary based on the particular muscle or nerve that the one or more electrodes are configured to stimulate. In the depicted example the stimulator 1230 includes a cuff electrode 1234 positioned at a distal end of the stimulator lead 1232, but the present disclosure is not limited thereto.

The stimulator controller 1210 may be coupled (e.g., operatively coupled) to at least some of the components of the implantable device 1200 (e.g., the receiver 1220, the one or more stimulators 1230, and/or the stimulator battery 1240) and configured to control at least some operations of the implantable device 1200. For example, as described in some detail above, the stimulator controller 1210 may be configured to control power transfer between the stimulator battery 1240 and the one or more stimulators 1230, and to cause the one or more stimulators 1230 to provide stimulation based on the physiological data sensed (e.g., measured) by the one or more sensors 1130.

For example, the stimulator controller 1210 may be configured to determine whether a stimulation start signal has been received by the implantable device 1200 (e.g., by the receiver 1220), and to cause the one or more stimulators 1230 to provide the stimulation in response to determining that the stimulation start signal has been received. The stimulation start signal may be a communication signal that was transmitted by the mouthpiece 1100 or the third device 1300, and which was transmitted in response to the mouthpiece 1100 or the third device 1300 respectively determining, based on the physiological data, that an apneic event or another medical event has occurred. In some embodiments, the stimulation start signal does not include the physiological data.

In some embodiments, the implantable device 1200 (e.g., the stimulator controller 1210) is configured to determine that the stimulation start signal is a qualified stimulation start signal that was generated by a qualified device (e.g., the mouthpiece 1100 or the third device 1300). For example, the implantable device 1200 may be configured to pair with the qualified device and to determine that the received stimulation start signal is a qualified stimulation start signal by determining that the stimulation start signal was generated by the paired qualified device. In some other embodiments, the qualified device may send the stimulation start signal as an encrypted message, and the implantable device 1200 may be configured to decode encrypted messages received from the qualified device and to determine that the received stimulation start signal is a qualified stimulation start signal when the implantable device 1200 is able to decode the stimulation start signal.

In some embodiments, the stimulator controller 1210 is configured to analyze physiological data, to determine, based on the physiological data, that an apneic event or another medical event has occurred, and to cause the one or more stimulators 1230 to provide the stimulation in response to determining that the apneic event or other medical event has occurred. The implantable device 1200 may be configured (e.g., by the receiver 1220) to receive a data signal including the physiological data from the mouthpiece 1100 or from the third device 1300, and the stimulator controller 1210 may be configured to be in communication with (e.g., to be communicatively coupled or couplable to) the receiver 1220 and to receive the physiological data from the receiver 1220.

In some embodiments, the implantable device 1200 may include one or more sensors configured to subcutaneously sense (e.g., measure) physiological data relating to the apnea or the other medical condition that the stimulation system 1000 is configured to treat, and/or the mouthpiece 1100 may include one or more stimulators configured to provide intraoral stimulation.

However, while subcutaneous sensors of an implantable device may be able to sense some kinds of physiological data, they cannot measure many kinds of physiological data with the same accuracy and ease as intraoral sensors of a mouthpiece can, and the intraoral sensors of the mouthpiece can measure some kinds of physiological data that subcutaneous sensors are generally unable (or unsuited) to measure. Additionally, while intraoral stimulation can provide some degree of treatment of apnea and other medical conditions, such stimulation generally cannot provide as precise, consistent, and efficient stimulation as subcutaneous stimulation can.

Accordingly, by sensing physiological data via the one or more sensors 1130 of the mouthpiece 1100, while providing stimulation via the one or more stimulators 1230 of the implantable device 1200 based on the physiological data sensed by the mouthpiece 1100, the stimulation system 1000 can more accurately determine the occurrence of apneic events and/or one or more other medical events, and provide more precise, consistent, and efficient stimulation to treat such event(s) in response to their occurrence. Additionally, by utilizing the mouthpiece 1100 to sense (and, in some embodiments, also to analyze) the physiological data, the complexity, size, cost, and invasiveness of the implantable device 1200 can be reduced compared to comparative embodiments where the implantable device 1200 includes sensors and a relatively large battery to provide additional power to operate the sensors and analyze the sensed physiological data.

In some embodiments, the implantable device 1200 may be configured to transmit (e.g., wirelessly transmit) a confirmation signal to the mouthpiece 1100 and/or to the third device 1300 indicating that stimulation has been provided. In some examples, the implantable device 1200 is configured to transmit the confirmation signal to the mouthpiece 1100 in response to receiving (e.g., from the mouthpiece 1100) the stimulation start signal and/or the data signal. The implantable device 1200 may be configured to transmit data to a programmer, for example, to the third device 1300, which may be used by the programmer. For example, the receiver 1220 may be part of a transceiver configured to both (e.g., sequentially, concurrently, or simultaneously) receive and transmit signals, or the implantable device 1200 may include a separate transmitter. In some examples, the mouthpiece 1100 includes a receiver or a transceiver configured to receive (e.g., wirelessly receive) signals from the implantable device 1200 and/or from the third device 1300.

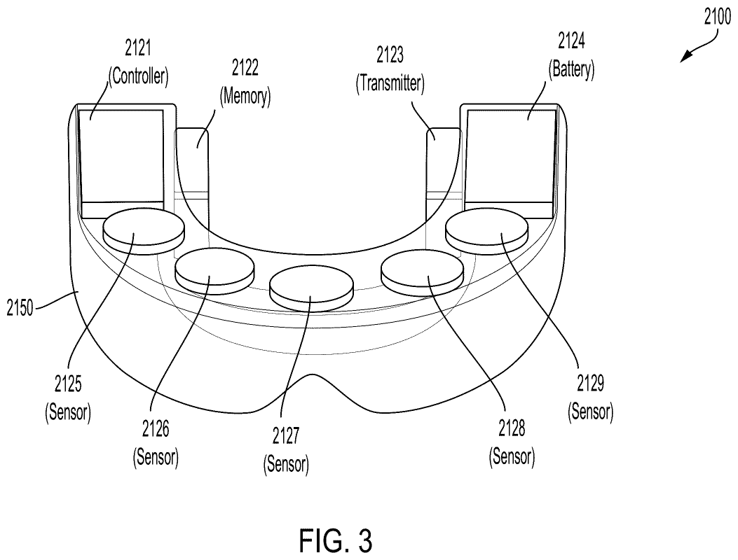

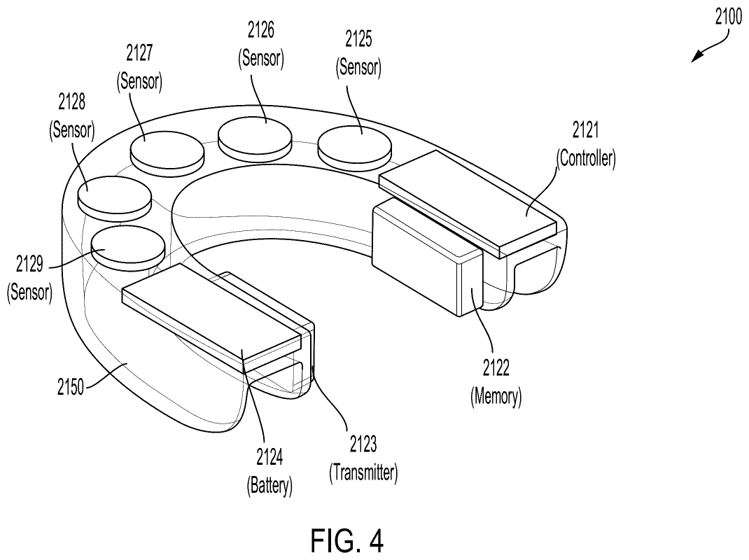

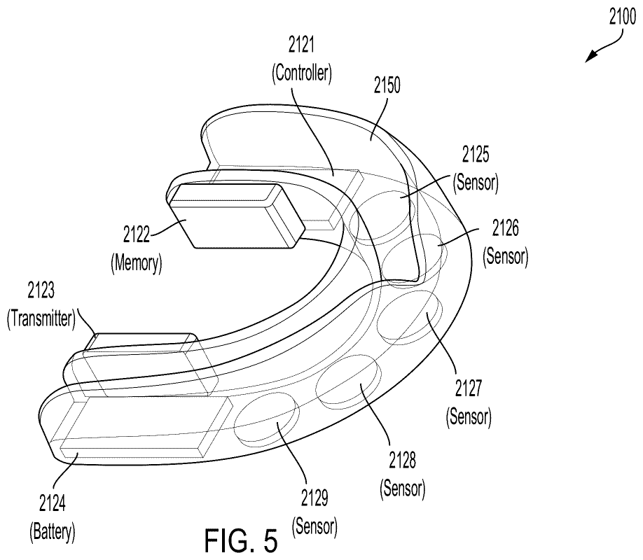

FIGS. 3 to 5 are perspective views, each depicting a mouthpiece 2100 of a stimulation system according to some embodiments. The mouthpiece 2100 may have some features similar to, or the same as, features illustrated, and described with respect to, the mouthpiece 1100 of the stimulation system 1000 of FIGS. 1 and 2. In some embodiments of the stimulation system 1000 of FIGS. 1 and 2, the mouthpiece 1100 may be the mouthpiece 2100 of FIGS. 3 to 5.

Referring concurrently to FIGS. 3 to 5, the mouthpiece 2100 may include a housing 2150 configured (e.g., shaped and sized) to be securely fitted to the bottom or roof of the mouth of a subject, for example, to be fitted over a bottom row of teeth or a top row of teeth of the subject. The housing 2150 may include (e.g., be) a compliant material, such as silicone rubber, and/or a rigid material. In some embodiments, the housing 2150 is hermetic and seals at least some of the components within the housing 2150 from the exterior of the housing 2150. The mouthpiece 2100 may include a plurality of components on and/or in the housing 2150, including a first component 2121, a second component 2122, a third component 2123, a fourth component 2124, a fifth component 2125, a sixth component 2126, a seventh component 2127, an eighth component 2128, and a ninth component 2129.

The first through ninth components 2121-2129 may each independently include (e.g., be) an electronic component, for example, a controller (e.g., microcontroller), electronics, a memory, an electrode, a transmitter (e.g., wireless communication transmitter), a battery, or a sensor. For example, as indicated in FIGS. 3-5, each of the fifth through ninth components 2125-2129 may include a sensor configured to sense (e.g., measure) physiological data, the first component 2121 may include a controller, the second component 2122 may include a memory, the third component 2123 may include a transmitter, and the fourth component 2124 may include a battery (e.g., a permanent battery or a rechargeable battery). However, the present disclosure is not limited thereto. For example, in some other embodiments the second and third components 2122 and 2123 may each include a sensor. Although the first through ninth components 2121-2129 are illustrated in FIGS. 3 to 5 as having certain shapes, positions, and orientations, the present disclosure is not limited thereto, and other shapes, positions, orientations, and number of components are within the scope of the present disclosure as would be recognized by a person skilled in the relevant art.

The operations and processes that are described herein as being performed by, or under the control of, a controller of the stimulation system (e.g., the controller of the mouthpiece, the implantable device, or the third device) may be performed in response to the controller executing computer-readable instructions that are stored in a memory and are associated with the performed operation of process. The controller may be coupled to the memory and configured to read and/or obtain the instructions stored in the memory for performing the operation(s) and/or process(es).

Nonlimiting and non-exhaustive embodiments of methods for providing stimulation to a subject for treating apnea and/or one or more other medical conditions will now be disclosed. Although some methods are disclosed herein, the present disclosure is not limited thereto. Rather, one or more suitable operations and processes of stimulation systems are disclosed herein, and those skilled in the art will understand and appreciate that the present disclosure includes all methods for providing stimulation for treating apnea and/or one or more other medical conditions that include any suitable number, combination, and order of these disclosed operations and processes to provide stimulation and/or to treat apnea and/or one or more other medical conditions.

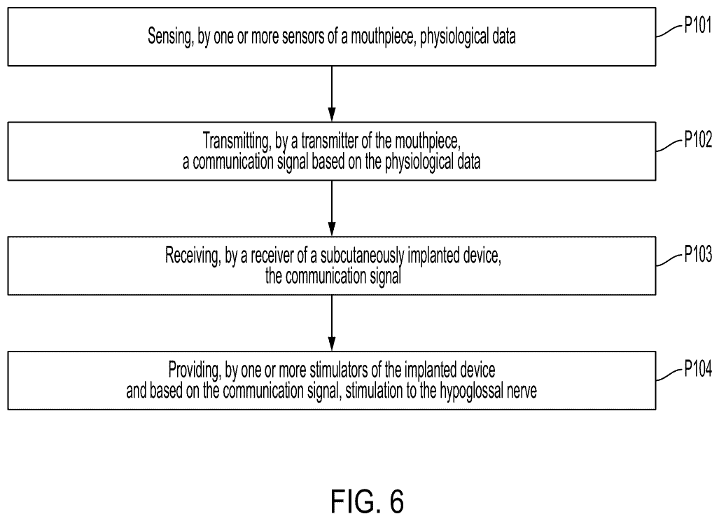

FIG. 6 depicts a method of providing stimulation for treating apnea according to some embodiments. In some embodiments, the method may be performed utilizing a suitable stimulation system disclosed herein.

Referring to FIG. 6, the method may include a first process P101 including sensing (e.g., measuring), by one or more sensors of a mouthpiece, physiological data of a subject; a second process P102 including transmitting (e.g., wirelessly transmitting), by a transmitter of the mouthpiece, a communication signal based on the physiological data; a third process P103 including receiving (e.g., wirelessly receiving), by a receiver of a subcutaneously implanted device, the communication signal; and a fourth process P104 including providing, by one or more stimulators of the implanted device and based on the communication signal, stimulation to the hypoglossal nerve (HGN). By stimulating the hypoglossal nerve, the subject's tongue may be moved and/or the subject's airway may be opened so that the subject is able to breathe.

In some embodiments, the method includes (e.g., after the first process P101) analyzing, by the mouthpiece (e.g., by a controller of the mouthpiece), the physiological data sensed by the one or more sensors to determine that an apneic event and/or one or more other medical events has occurred, and the second process P102 may include transmitting the communication signal as a stimulation start signal. The fourth process P104 may include providing stimulation to the HGN in response to receiving the stimulation start signal.

In some other embodiments, the second process P102 includes transmitting the communication signal as a data signal that includes at least some of the physiological data, and the fourth process P104 includes analyzing, by the implanted device (e.g., by a controller of the implanted device), the at least some of the physiological data to determine that an apneic event and/or one or more other medical events has occurred. The stimulation to the HGN may then be provided in response to determining that the apneic event and/or the one or more other medical events has occurred.

In some embodiments, the fourth process P104 may include causing, by a controller of the implanted device and based on the communication signal, power to be provided by a battery of the implanted device to the one or more stimulators to provide the stimulation.

In some embodiments, the physiological data includes at least one data (e.g., one data, two data, three data, four data, etc.) of an ultrasound measurement, a moisture measurement, a temperature measurement, an inertia measurement, an electrocardiogram, an oxygen-blood level, an airflow measurement, a sound measurement, a pressure measurement, an actigraphy-based measurement, or a brain activity measurement. For example, the physiological data may include at least one data of an ultrasound measurement, a moisture measurement, a temperature measurement, an inertia measurement, or an electrocardiogram. In some embodiments, the physiological data includes at least two data (e.g., two data, three data, four data, etc.) of an ultrasound measurement, a moisture measurement, a temperature measurement, an inertia measurement, an electrocardiogram, an oxygen-blood level, an airflow measurement, a sound measurement, a pressure measurement, an actigraphy-based measurement, or a brain activity measurement.

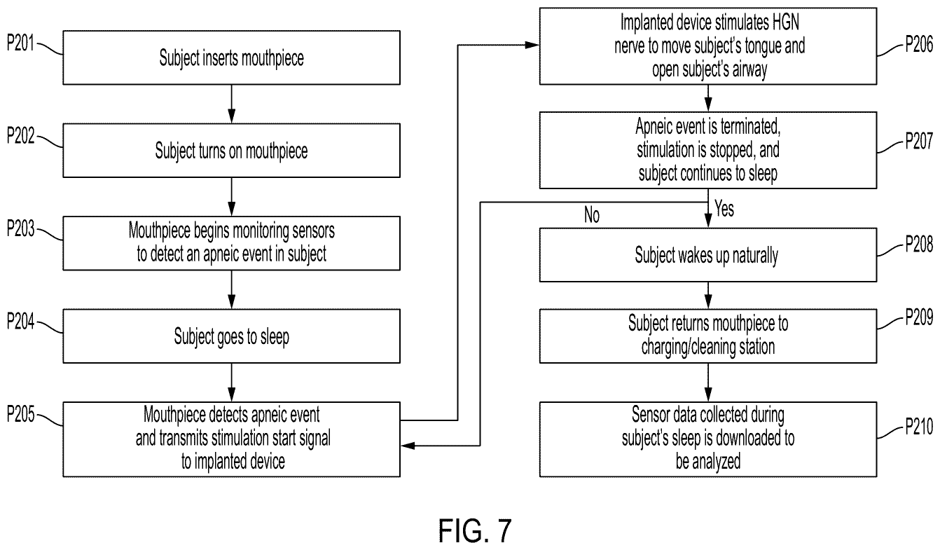

The stimulation systems and methods for providing stimulation may be utilized by a subject (e.g., a human patient) to treat apnea and/or one or more other medical conditions while the subject is asleep. FIG. 7 depicts a method for providing stimulation for treating apnea and/or one or more other medical conditions according to some embodiments. The method of FIG. 7 may include some features (e.g., processes) similar to, or the same as, the features of the method of FIG. 6. In some embodiments, the method of FIG. 7 may be performed utilizing a suitable stimulation system disclosed herein.

Referring to FIG. 7, the method may include a first process P201 including the subject intraorally inserting a mouthpiece, and a second process P202 including turning on the mouthpiece. In some embodiments, the mouthpiece may be turned on wirelessly, for example, by a patient controller (e.g., the third device 1300 described herein with reference to FIG. 1) that is to be in communication with the mouthpiece. The method may also include a third process P203 including monitoring, by the mouthpiece, sensors of the mouthpiece to detect an apneic event in the subject; a fourth process P204 including the subject going to sleep; a fifth process P₂O₅ including detecting, by the mouthpiece, an apneic event and transmitting a stimulation start signal to a subcutaneously implanted device in the subject; a sixth process P206 including stimulating, by the implanted device, a hypoglossal nerve (HGN) of the subject to cause the subject's tongue to move and/or to open the subject's airway so that the subject begins breathing; and a seventh process P207 in which the apneic event is terminated, stimulation by the implanted device stops, and the subject continues to sleep.

During an eighth process P208, if the subject does not wake up, then the mouthpiece continues to monitor its sensors and detects a subsequent apneic event in the fifth process P205, and the fifth and sixth processes P205 and P206 are repeated. However, when the subject wakes up during the eighth process P208, then the subject returns the mouthpiece to a charging/cleaning station during a ninth process P209. Reference to the subject waking up refers to the subject waking up permanently from his or her sleep, as opposed to temporarily being awakened before going back to sleep, which may occur in response to the stimulation being provided during the sixth process P206. The charging/cleaning station may be a station for holding the mouthpiece while the subject is not sleeping, and may be configured to electrically couple to the mouthpiece to charge the mouthpiece (e.g., to charge a battery of the mouthpiece). The charging/cleaning station may also be configured to clean (e.g., sanitize) the mouthpiece.

The method may also include a tenth process P210 including downloading (e.g., downloading to the charging/cleaning station and/or to the patient controller) the physiological data sensed by the one or more sensors while the subject was asleep. The physiological data may be analyzed to determine, for example, characteristics of apneic events experienced by the subject, which can be utilized to better detect apneic events when the subject goes to sleep with the mouthpiece in the future. For example, the characteristics may include the frequency and/or pattern of apneic events while the subject was asleep, what changes in physiology most reliably accompany and/or indicate apneic events, etc. As disclosed herein, determining whether an apneic event has occurred, for example, by a controller of the mouthpiece, the implantable device, or the third device, may be based at least in part on the collected data and/or the analysis of the collected data. For example, the mouthpiece may include a memory configured to store the collected data and/or the analysis of the collected data.

The stimulation systems and/or any other relevant devices or components (e.g., the mouthpiece, the implantable device, the third device, and respective components thereof) according to embodiments of the present disclosure described herein may be implemented utilizing any suitable hardware, firmware (e.g. an application-specific integrated circuit), software, or a combination of software, firmware, and hardware. For example, the one or more suitable components of the mouthpiece, the implantable device, and/or the third device may be formed on one integrated circuit (IC) chip or on separate IC chips. Further, the one or more suitable components of the mouthpiece, the implantable device, and/or the third device may be implemented on a flexible printed circuit film, a tape carrier package (TCP), a printed circuit board (PCB), or formed on one substrate. Further, the one or more suitable components of the mouthpiece, the implantable device, and/or the third device may be a process or thread, running on one or more processors, in one or more computing devices, executing computer program instructions and interacting with other system components for performing the one or more suitable functionalities described herein.

The computer program instructions may be stored in a memory which may be implemented in a computing device utilizing a standard memory device, such as, for example, a random access memory (RAM). The computer program instructions may also be stored in other non-transitory computer readable media such as, for example, a CD-ROM, flash drive, and/and/or the like. Also, a person of skill in the art should recognize that the functionality of one or more suitable computing devices may be combined or integrated into a single computing device, or the functionality of a particular computing device may be distributed across one or more other computing devices without departing from the scope of the example embodiments of the present disclosure.

It should also be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the drawings, it should be understood by those of ordinary skill in the art that one or more suitable changes in form and details may be made therein without departing from the spirit and scope as defined by the following claims and equivalents thereof.

What is claimed is:

1. A stimulation system, comprising:
a mouthpiece comprising one or more sensors to intraorally sense physiological data of a subject; and
a subcutaneously implantable device to be in communication with the mouthpiece and comprising one or more stimulators to stimulate a hypoglossal nerve of the subject to treat sleep apnea.

2. The stimulation system of claim 1, wherein the one or more sensors comprise at least one of an ultrasound sensor, a moisture sensor, a temperature sensor, an inertial sensor, an electrocardiogram sensor, a pulse oximeter, an airflow sensor, a microphone, a pressure sensor, an actigraphy sensor, or an electroencephalogram sensor.

3. The stimulation system of claim 1, wherein the one or more sensors comprise at least one of an ultrasound sensor, a moisture sensor, a temperature sensor, an inertial sensor, or an electrocardiogram sensor.

4. The stimulation system of claim 1, wherein the one or more sensors comprise at least two of an ultrasound sensor, a moisture sensor, a temperature sensor, an inertial sensor, an electrocardiogram sensor, a pulse oximeter, an airflow sensor, a microphone, a pressure sensor, an actigraphy sensor, or an electroencephalogram sensor.

5. The stimulation system of claim 1, wherein the mouthpiece comprises a transmitter to wirelessly transmit a communication signal based on the physiological data sensed by the one or more sensors, and the subcutaneously implantable device comprises a receiver to wirelessly receive the communication signal.

6. The stimulation system of claim 5, wherein the mouthpiece comprises:
a battery; and
a controller to determine, based on the physiological data, that an apneic event of the subject has occurred, and to cause the receiver to transmit the communication signal as a stimulation-start signal in response to determining that the apneic event has occurred.

7. The stimulation system of claim 5, wherein the mouthpiece is to transmit the communication signal as a data signal comprising at least some of the physiological data, and the subcutaneously implantable device comprises a controller to determine, based on the physiological data, that an apneic event of the subject has occurred and to stimulate the nerve or the muscle in response to determining that the apneic event has occurred.

8. The stimulation system of claim 1, wherein the one or more stimulators are to stimulate at least a hypoglossal nerve.

9. The stimulation system of claim 8, wherein the subcutaneously implantable device comprises a housing comprising one or more electronic components, and
wherein the one or more stimulators comprises:
a stimulator lead extending, at a proximal end of the stimulator lead, from the housing; and
a cuff electrode positioned along the stimulator lead and to stimulate the hypoglossal nerve.

10. The stimulation system of claim 1, wherein the subcutaneously implantable device comprises a battery to provide power to the one or more stimulators.

11. The stimulation system of claim 10, wherein the subcutaneously implantable device comprises:
a receiver to wirelessly receive a communication signal from the mouthpiece; and
a controller communicatively coupled to the receiver and to cause power to be provided from the battery to the one or more stimulators in response to the receiver receiving the communication signal.

12. A stimulation system, comprising:
a subcutaneously implantable device comprising:
one or more stimulators to provide nerve stimulation to a hypoglossal nerve of a subject;
a battery to provide power to the one or more stimulators; and
a receiver device to wirelessly receive a communication signal,
wherein the subcutaneously implantable device is to provide the nerve stimulation, via the one or more stimulators, based on the communication signal.

13. The stimulation system of claim 12, wherein the subcutaneously implantable device comprises a controller communicatively coupled to the receiver device and to controllably cause the power to be provided from the battery to the one or more stimulators.

14. The stimulation system of claim 13, wherein the communication signal is a data signal comprising physiological data, and the controller is to cause the power to be provided from the battery to the one or more stimulators in response to determining, by the controller and based on the physiological data, that an apneic event of the subject has occurred.

15. The stimulation system of claim 13, wherein the communication signal is a stimulation-start signal, and the controller is to cause the power to be provided from the battery to the one or more stimulators in response to the receiver device receiving the stimulation-start signal.

16. The stimulation system of claim 15, wherein the stimulation-start signal is a qualified stimulation-start signal that the controller is to recognize as being generated by a qualified mouthpiece device.

17. The stimulation system of claim 12, wherein the subcutaneously implantable device comprises a housing comprising the battery and the receiver device, and
wherein the one or more stimulators comprises a stimulator lead extending, at a proximal end of the stimulator lead, from the housing and one or more electrodes positioned along the stimulator lead and to stimulate the hypoglossal nerve.

18. The stimulation system of claim 12, further comprising a mouthpiece comprising one or more sensors to intraorally sense physiological data of the subject.

19. The stimulation system of claim 18, wherein the mouthpiece comprises a transmitter to wirelessly transmit the communication signal to the receiver device of the subcutaneously implantable device.

20. The stimulation system of claim 19, wherein the mouthpiece comprises a controller to determine, based on the physiological data, whether an apneic event of the subject has occurred, and to transmit, by the transmitter, a stimulation-start signal to the receiver device in response to determining that the apneic event has occurred.

21. The stimulation system of claim 18, wherein the one or more sensors comprise at least one of an ultrasound sensor, a moisture sensor, a temperature sensor, an inertial sensor, or an electrocardiogram sensor.

22. The stimulation system of claim 12, wherein the battery is a rechargeable battery, and
wherein the subcutaneously implantable device further comprises a power receiver coil electrically coupled to the rechargeable battery and configured to wirelessly receive power.

23. A method of providing stimulation for treating apnea, the method comprising:
wirelessly receiving, by a receiver of a subcutaneously implanted device, a communication signal; and
providing stimulation, by one or more stimulators of the subcutaneously implanted device and based on the communication signal, to a hypoglossal nerve of a subject.

24. The method of claim 23, comprising causing, by a controller of the subcutaneously implanted device and based on the communication signal, power to be provided by a battery of the subcutaneously implanted device to the one or more stimulators to provide the stimulation.

25. The method of claim 24, wherein the communication signal is a data signal comprising physiological data, and
wherein the method comprises determining, by the controller and based on the physiological data, that an apneic event of the subject has occurred, and providing the stimulation in response to the determining that the apneic event has occurred.

26. The method of claim 24, wherein the communication signal is a stimulation-start signal, and
wherein the method comprises providing the stimulation in response to the subcutaneously implanted device receiving the stimulation-start signal.

27. The method of claim 23, comprising intraorally sensing, by one or more sensors of a mouthpiece, physiological data of the subject.

28. The method of claim 27, comprising wirelessly transmitting, by a transmitter of the mouthpiece, the communication signal.

29. The method of claim 28, comprising determining, by a controller of the mouthpiece and based on the physiological data, that an apneic event of the subject has occurred, and transmitting the communication signal as a stimulation-start signal in response to the controller determining that the apneic event has occurred.

30. The method of claim 27, wherein the physiological data comprises at least one data of an ultrasound measurement, a moisture measurement, a temperature measurement, an inertia measurement, an electrocardiogram, an oxygen-blood level, an airflow measurement, a sound measurement, a pressure measurement, an actigraphy-based measurement, or a brain activity measurement.

31. The method of claim 27, wherein the physiological data comprises at least one data of an ultrasound measurement, a moisture measurement, a temperature measurement, an inertia measurement, or an electrocardiogram.

32. The method of claim 27, wherein the physiological data comprises at least two data of an ultrasound measurement, a moisture measurement, a temperature measurement, an inertia measurement, an electrocardiogram, an oxygen-blood level, an airflow measurement, a sound measurement, a pressure measurement, an actigraphy-based measurement, or a brain activity measurement.
